(19) United States
(12) Patent Application Publication
(10) Pub. No.: US 2013/0189773 A1
(43) Pub. Date: Jul. 25, 2013

(54) PRODUCTION OF HYPOTHIOCYANITE FROM HALOGENATED CELLS

(71) Applicant: Michael T. Ashby, Norman, OK (US)

(72) Inventor: Michael T. Ashby, Norman, OK (US)

(21) Appl. No.: 13/357,219

(22) Filed: Jan. 24, 2012

(51) Int. Cl. A23B 4/20; C12N 5/04; A23B 7/154; C12N 5/07

(52) U.S. Cl. 435/325; 435/410; 426/335; 426/332

(57) ABSTRACT

Methods of producing antimicrobial compositions include reacting halogenated eukaryotic cells with thiocyanate to produce the antimicrobial compound hypothiocyanite.

### STATEMENT REGARDING FEDERALLY SPONSORED RESEARCH OR DEVELOPMENT

This invention was made with government support under Contract Number CHE-0911328 awarded by the National Science Foundation (NSF). The government has certain rights in the invention.

### CROSS REFERENCE TO RELATED APPLICATIONS

Not Applicable.

### BACKGROUND OF THE INVENTION

1. Field of the Inventive Concept(s)

The presently disclosed and claimed inventive concept(s) relates generally to production of antimicrobial compositions, and in particular, but not by way of limitation, to production of hypothiocyanite from halogenated cells.

2. Description of the Background Art

There has been an alarming rise in the occurrence of disease-causing microbes that have become resistant to drug therapy. This antibiotic resistance is largely due to increased use of antibiotics and the remarkable resiliency of pathogenic organisms that have developed ways to survive the drugs that are meant to target them. Pneumonia, tuberculosis, malaria, and ear infections are just a few of the diseases that have become difficult to treat with our current arsenal of antibiotic drugs. All FDA-approved antibiotics that are currently in use are organic molecules. The principal endogenous non-immunological anti-microbial agent in the secretions of human exocrine glands such as saliva, tears, and milk is an inorganic electrophilic thiocyanating agent commonly referred to as “hypothiocyanite” (OSCN⁻).

An enzymatic method for synthesizing hypothiocyanite is known, and there are certain products on the market that are based upon this technology. The only conventional (non-enzymatic) chemical method for synthesizing hypothiocyanite is based upon the synthesis of thiocyanogen in halogenated hydrocarbon solvents using salts of lead, and thus this method utilizes reagents that are highly toxic and/or carcinogenic.

While the antimicrobial activities of generating hypothiocyanite have been recognized, current methods of generating hypothiocyanite utilize expensive enzyme-based systems; for example, the BIOTENE® line of dental hygiene products utilize lactoperoxidase and glucose oxidase enzyme systems to generate hypothiocyanite.

The inventor's earlier patent, U.S. Pat. No. 7,238,334, issued Jul. 14, 2005 and expressly incorporated herein by reference in its entirety, is directed to a method of synthesizing hypothiocyanite in a solution by combining hypohalous acid and thiocyanate under conditions of turbulent mixing to produce a mixture comprising hypothiocyanite. While this method provides a solution effective for disinfectant applications, due to the short-lived nature of hypothiocyanite in solution, the '334 patent does not provide methods of providing anti-microbial protection to cells, tissues and/or foodstuffs (where the rate of decomposition of hypothiocyanite competes with its rate of penetration into the foodstuffs).

Thus, there is a need in the art for new and improved and inexpensive antimicrobial treatment of foodstuffs. It is to said antimicrobial treatment methods that the presently disclosed and claimed inventive concept(s) is directed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates that small molecular, macromolecular and cellular chloramines react with SCN⁻ to give OSCN⁻. A) Reaction of taurine chloramine (TauCl) with excess SCN⁻ gives a quantitative yield of taurine (Tau) and OSCN⁻. B) Reaction of ubiquitin (Ub) with HOCl and subsequent reaction with SCN⁻ gives OSCN⁻. C) Chlorination of E. coli (MG1655) cells with HOCl, pelleting by centrifugation, suspension in phosphate buffer (to give bacterial cells with “chlorine cover”), reaction of the cells with SCN⁻, and filter sterilization through a 0.2 μM filter yields OSCN⁻.

FIG. 2 contains kinetic traces at 340 nm depicting the GOR-catalyzed reduction of GSSG by NADPH. Conditions: (Control) [GSSG]ₒ=1 mM, [NADPH]ₒ=0.1 mM, [GOR]=0.025 U/mL, [iP]=0.1 M, pH 7.4 and T=25° C.; (SCN⁻ treated) GOR incubated with 40 μM HOCl for 20 minutes and then treated with 160 μM SCN⁻ for another 20 minutes. (HOCl modified) GOR incubated with 40 μM HOCl for 20 minutes and then treated with iP for an additional 20 minutes.

FIG. 3(A)-(C) contains fluorescence images of A549 cells that have been untreated (FIG. 3A), treated with 50 μM HOCl (FIG. 3B), or treated with 50 μM SCN⁻ subsequent to treatment with 50 μM HOCl (FIG. 3C). In each experiment, A549 cells were incubated with 50 μM HOCl in RPMI culture medium for 20 min at 37° C. and washed with the medium to remove residual HOCl; the cells were then incubated for an additional 20 minutes at 37° C. with 50 μM SCN⁻ and washed again with the medium. FIG. 3D illustrates the structure of the rhodamine-hydroxymic acid-based probe utilized in FIGS. 3A-C.

FIG. 4 graphically illustrates the percent of live, necrotic, and apoptotic cells (essentially zero) after following the protocol that was used to investigate the reaction of intracellular chloramines with SCN⁻ (FIG. 3). A549 cells were untreated (A), treated with 50 μM HOCl (B), treated with 50 μM SCN⁻ followed by treatment with 50 μM HOCl, (C), or treated with 50 μM HOCl followed by treatment 30 minutes later with 50 μM SCN⁻ (D).

### DETAILED DESCRIPTION OF THE INVENTIVE CONCEPT(S)

Before explaining at least one embodiment of the inventive concept(s) in detail by way of exemplary drawings, experimentation, results, and laboratory procedures, it is to be understood that the inventive concept(s) is not limited in its application to the details of construction and the arrangement of the components set forth in the following description or illustrated in the drawings, experimentation and/or results. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed and claimed inventive concept(s) shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2nd ed., Cold Spring Harbor Laboratory Press, Cold Spring Harbor, N.Y. (1989) and Coligan et al. Current Protocols in Immunology (Current Protocols, Wiley Interscience (1994)), which are incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

All patents, published patent applications, and non-patent publications mentioned in the specification are indicative of the level of skill of those skilled in the art to which this presently disclosed and claimed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the inventive concept(s) as defined by the appended claims.

As utilized in accordance with the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

The term “about” is used to indicate that a value includes the inherent variation of error for the device, the method being employed to determine the value and/or the variation that exists among study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “antimicrobial” as utilized herein refers to a substance capable of killing different forms of microbial living organisms. Examples of antimicrobial substances include, but are not limited to, germicides, antibiotics, antibacterials, antivirals, antifungals, antiprotoas, antiparasites, and the like.

The presently disclosed and claimed inventive concept(s) is directed to an antimicrobial treatment method for eukaryotic cell(s), such as but not limited to, foodstuffs. Non-limiting examples of foodstuffs that may be treated in accordance with the presently disclosed and claimed inventive concept(s) include, but are not limited to, animal protein and meat, vegetables, fruit, byproducts thereof, and the like. The presently disclosed and claimed inventive concept(s) overcomes the defects and disadvantages of the prior art by replacing expensive enzymatic systems of generating hypothiocyanite with a simple, inexpensive chemical treatment that results in generation of hypothiocyanite. In the method, cells treated with a halogenating agent are subsequently treated with thiocyanate, whereby hypothiocyanite is produced.

Currently, foodstuffs are being treated with hypochlorous acid (i.e., by spraying hypochlorous acid onto meat or dipping fruit or vegetables into hypochlorous acid). While this method provides some antimicrobial properties, hypochlorite only reacts with a surface of the tissue, and thus does not penetrate into the cells. Therefore, hypochlorite treatment does not affect any potential subcutaneous microbial infection present in the tissue. The presently disclosed and claimed inventive concept(s) combines treatment with a halogenating agent (such as but not limited to, hypochlorous acid) with a subsequent treatment with thiocyanate to produce hypothiocyanite. The combination of these two treatments provides two different mechanisms of antimicrobial action, and said mechanisms exhibit cooperative and synergistic properties. Moreover, the hypothiocyanite is generated inside of the cells, and thus provides an internal mechanism of antimicrobial treatment for cells/tissues.

In addition, treatment with thiocyanate will additionally assist with removal of undesired smells/odors as well as wash water contamination resulting from prior treatment with halogenating agents. The current single treatment with certain halogenating agents, such as but not limited to hypochlorite, results in an undesired “chlorine” smell attached to the surface of the treated cells; in addition, significant amounts of hypochlorite generally remain in the water after washing or spraying the foodstuffs. In contrast, treatment with thiocyanate removes hypochlorite from the water, and thus no electrophilic chlorinating agent is left—only hypothiocyanite remains, and is innocuous to man. In addition, the reaction of thiocyanate with hypochlorite will remove the undesired chlorine smell from the treated foodstuffs.

Turning now to particular embodiments of the presently disclosed and claimed inventive concept(s), a method of producing hypothiocyanite is provided. In the method, at least one halogenated eukaryotic cell, such as but not limited to, a halogenated eukaryotic tissue and/or foodstuff (e.g., animal protein, animal meat, vegetables, fruit, byproducts thereof, etc.) is produced by treating the eukaryotic cell with a halogenating agent; then the at least one halogenated eukaryotic cell is reacted with thiocyanate. The thiocyanate reacts with the halogen present on a surface of the eukaryotic cell to produce hypothiocyanite.

The halogenating agent may be any halogenated agent known in the art or otherwise contemplated herein. For example but not by way of limitation, the halogenating agent may comprise hypochlorous acid, molecular chlorine, chloramines, and combinations or derivatives thereof. Following treatment with halogenating agent, immobilized oxidant is produced on the surface of the cell.

In contrast to the large excess of thiocyanate, the alkaline medium, and the rapid (turbulent) mixing conditions that are required to produce quantitative amounts of hypothiocyanite from hypochlorite, the immobilized oxidant produced on the surface of the cell following treatment by halogenating agents reacts under more controlled conditions to produce high yields of hypothiocyanite without the need of special precautions (such as high pH and rapid mixing), thereby affording practical implementation.

In certain instances, the reaction of the at least one halogenated eukaryotic cell with thiocyanate will substantially remove an odor associated with the halogen present on the surface of the at least one eukaryotic cell.

Another embodiment of the presently disclosed and claimed inventive concept(s) is directed to a method of providing antimicrobial protection to at least one eukaryotic cell, such as but not limited to, a eukaryotic tissue and/or foodstuff (e.g., animal protein, animal meat, vegetables, fruit, byproducts thereof, etc.). In the method, at least one eukaryotic cell having immobilized oxidant on a surface thereof (e.g., chloramines produced by prior reaction with hypochlorite) is treated/reacted with thiocyanate. The at least one eukaryotic cell has been treated with a halogenating agent (as described in detail herein above) to provide immobilized oxidant on the surface thereof. The reaction of thiocyanate with the at least one eukaryotic cell with immobilized oxidant on the surface thereof results in the production of hypothiocyanite.

In certain instances, the reaction of the at least one halogenated eukaryotic cell with thiocyanate will substantially remove an odor associated with the immobilized oxidant present on the surface of the at least one eukaryotic cell.

### EXAMPLES

Examples are provided hereinbelow. However, the presently disclosed and claimed inventive concept(s) is to be understood to not be limited in its application to the specific experimentation, results and laboratory procedures described herein. Rather, the Examples are simply provided as one of various embodiments encompassed by the scope of the presently disclosed and claimed inventive concept(s) and are meant to be exemplary, not exhaustive.

### Example 1

Myeloperoxidase (MPO) is released intraphagosomally and extracellularly by activated neutrophils, monocytes and some macrophages, and said release can initiate and promote host tissue damage. MPO catalyzes the oxidation of Cl⁻ (or other halogen) and SCN⁻ by H₂O₂ with the production of the potent oxidant hypochlorous acid (HOCl) and less reactive hypothiocyanous acid (HSCN), respectively (Hampton et al., 1998). HOCl production appears to play a beneficial role in human immune defense, particularly in the phagolysosome, where it is believed to kill infectious agents. HOCl modifies chemical functional groups, causing wholesale cellular damage and eventual death. However, collateral host tissue damage by extracellular HOCl is implicated in the pathogenesis of many human inflammatory diseases, including but not limited to, atherosclerosis, asthma, cystic fibrosis, periodontal disease, rheumatoid arthritis, kidney disease, and some cancers. In contrast to potentially deleterious effects of HOCl, HOSCN is relatively innocuous towards host tissues. The amounts of HOCl and HOSCN that are produced by the MPO enzyme system are dictated by the bioavailabilities of the substrates Cl⁻ and SCN⁻ and their relative reactivities.

A hierarchy of reactivity exists for HOCl, with sulfur-containing moieties amongst the most efficient scavengers, but other chemical moieties are lesser targets, including amines that form chloramine derivatives upon reaction with HOCl. Glutathione (GSH), the predominant non-protein thiol in eukaryotes and some prokaryotes, protects the cytosol of mammalian cells from damage by HOCl and other oxidants. It has been suggested that the inorganic sulfur-containing ion thiocyanate (SCN⁻) may serve a similar role as a sequestering agent of HOCl (Ashby et al., 2004). Given the high reactivity of SCN⁻ toward HOCl, a reaction that produces relatively benign hypothiocyanite (OSCN⁻; Nagy et al., 2006), the presence of SCN⁻ prevents HOCl-induced cell death of prokaryotic and eukaryotic cells. However, this Example demonstrates that SCN⁻ was also capable of resuscitating mammalian cells that have been subjected to otherwise lethal amounts of HOCl. At the molecular level, chloramines react with SCN⁻ to produce OSCN⁻ (Xulu et al., 2010). At the protein level, SCN⁻ returned function to key enzymes like glutathione reductase (a cysteine-active site enzyme that is responsible for maintaining GSH redox homeostasis) that have been damaged by HOCl. At the cellular level, chloramines were repaired and viability improved upon treatment of HOCl-damaged mammalian cells with SCN⁻. These results demonstrate that perfusion of SCN⁻-containing physiological fluids to tissues that have been damaged by inflammatory response may be remedial.

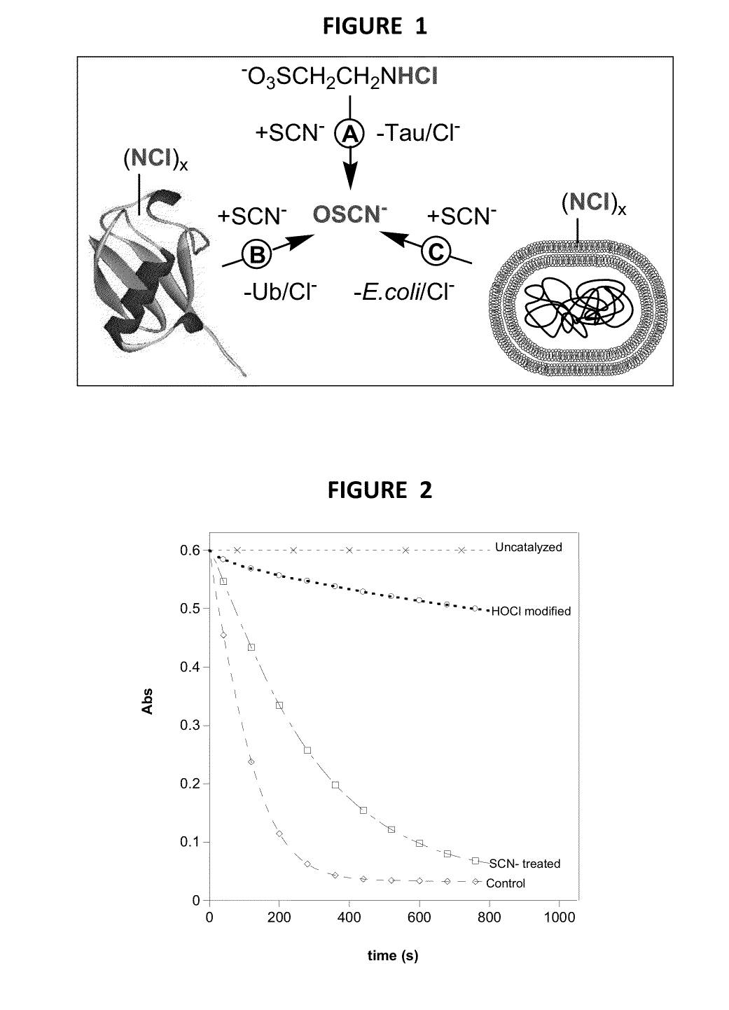

FIG. 1 demonstrates that small molecular, macromolecular and cellular chloramines react with SCN⁻ to give OSCN⁻. Proteins may be one of the principal initial targets of HOCl in vivo, and chloramines are a significant product. Chloramines eventually decompose to irreversibly damage proteins. The inventor has previously demonstrated (Xulu et al., 2010) that SCN⁻ reacts efficiently with chloramines in small molecules and proteins, and with the “chlorine cover” on the surface of E. coli cells to give OSCN⁻ and the parent amine. Remarkably, OSCN⁻ reacts faster than SCN⁻ with chloramines.

FIG. 2 illustrates the inhibition of GSH reductase (GOR) by HOCl and repair by SCN⁻. Glutathione (GSH) is a major non-protein thiol in cells (up to mM concentrations have been reported in some cells). One of the cellular functions of GSH is to serve as an antioxidant, and thus GSH is essential for cell survival, especially during conditions of oxidative stress. Many of the important residues in the active site of GOR, including two cysteines (Cys⁵⁸ and Cys⁶³), histidine (His⁴⁶⁷) and tyrosine (Tyr¹⁹⁷), are susceptible to HOCl attack. Oxidation of these residues results in the inhibition of enzyme activity. Treatment of GOR (˜1.3 nM) with 40 μM HOCl for 20 minutes resulted in severe loss (˜82%) of enzyme activity within 13 minutes (FIG. 2). Surprisingly, when the HOCl-modified GOR was subsequently treated with 160 μM SCN⁻ for 20 minutes, most of the enzyme activity (˜89%) was recovered. Importantly, since only the sulfenic acid and its corresponding thiosulfenate ester are known to react with SCN⁻, this observation suggests that Cys⁵⁸ and Cys⁶³ are not over-oxidized by the excess HOCl. This is an important observation that suggests that hydrophobicity around the active site prevents involvement of water in the oxidation process, a requirement for oxidation beyond the disulfide oxidation state. Instead, it is proposed that His⁴⁶⁷ is oxidized to a chloramine and subsequently reduced back by SCN⁻.

### Example 2

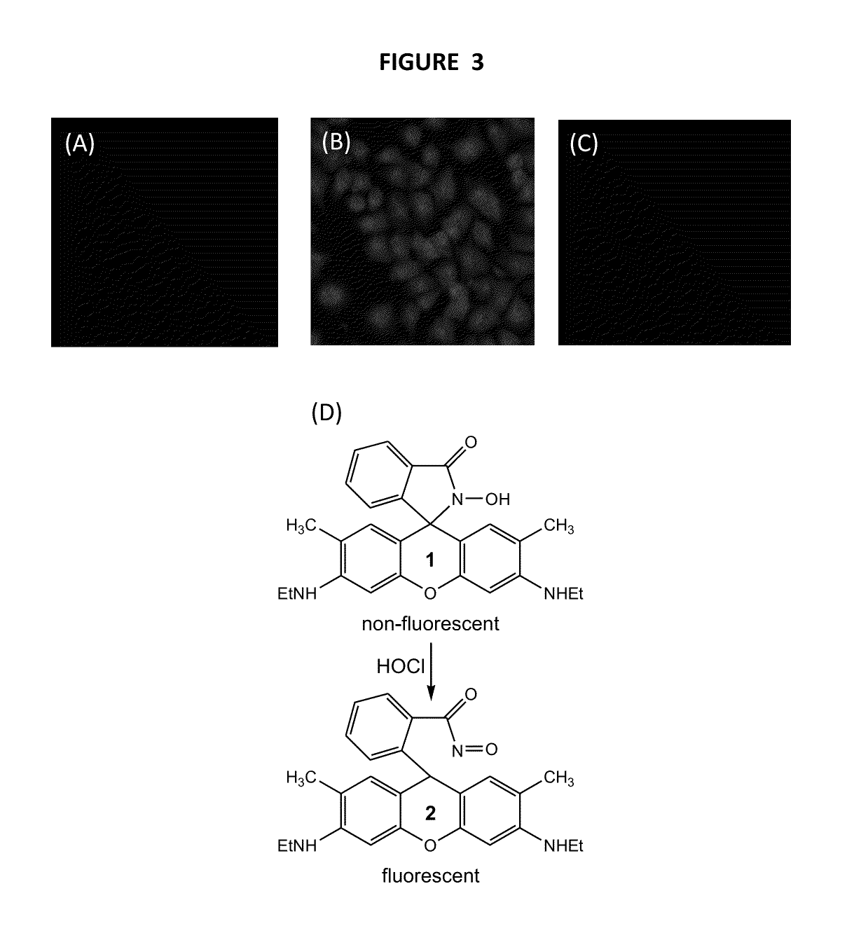

FIG. 3 demonstrates that HOCl-produced intracellular chloramines in mammalian cells react with SCN⁻. This observation is unexpected, because it was thought that intracellular amines that react with hypochlorite to produce chloramines are protected by cytosolic glutathione (with its more reactive moiety relative to amines). Recently, Tae et al. (2009) described the synthesis of a new, highly selective and sensitive fluorescent probe for HOCl detection in aqueous media. The new probe is rhodamine-hydroxymic acid-based (FIG. 3D) and is designed in a way that HOCl selectively and irreversibly oxidizes the hydroxamic acid unit of the probe, resulting in the ring opening and the production of a highly fluorescent acyl nitroso compound (2). Although originally designed to detect HOCl, the inventor has discovered that compound (1) reacts with all electrophilic halogenating agents (including chloramines). Compound (1) has been employed to probe the production of chloramines in the A549 carcinomic human alveolar basal epithelial cell line. A549 cells that have not been treated with HOCl did not fluoresce when treated with compound (1) (FIG. 3A). Cells that were treated with HOCl and subsequently with the probe fluoresced (FIG. 3B). If the cells were treated with HOCl in the presence of SCN⁻, they did not fluoresce, presumably because SCN⁻ reacts with the HOCl before it reacts with the cells to form chloramines (data not shown). Importantly, cells that were treated with HOCl and subsequently with SCN⁻ do not fluoresce when treated with the probe (FIG. 3C).

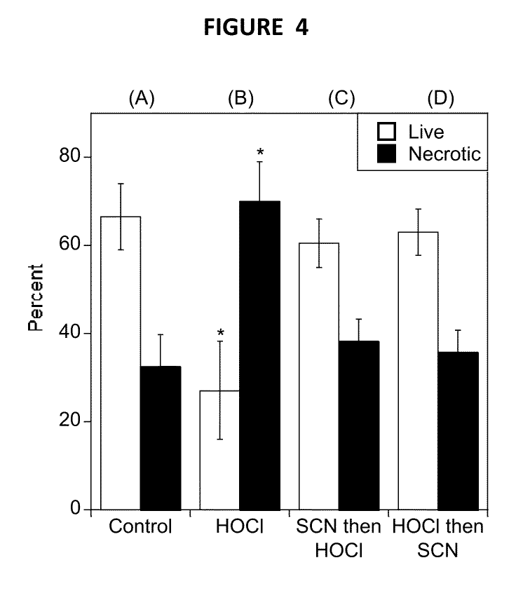

HOCl-damaged mammalian A549 cells were resuscitated with SCN⁻. Using the same protocol that was used to investigate the reaction of intracellular chloramines with SCN⁻ (FIG. 3), flow cytometry was employed to investigate the percent of live, necrotic, and apoptotic cells (FIG. 4). The amount of HOCl that was employed was sufficient to kill roughly half of the cells (FIG. 4B). As has been observed previously, SCN⁻ was an efficient scavenger of HOCl when present in sufficient amounts before the HOCl was added (FIG. 4C). Remarkably, if the same amount of SCN⁻ was added to the HOCl-damaged cells within 30 minutes (FIG. 4D, cf. FIG. 3C), the cells were resuscitated. However, if the interval for addition of SCN⁻ was 1 hour or more, recovery was not observed (data not shown). The latter results are consistent with the known timeframe for decomposition of protein chloramines to radicals and aldehydes. Note that under the conditions of these experiments, essentially no apoptotic cells were observed.

### Summary of Examples

In summary, these Examples demonstrate that: (1) chloramines that are produced in cells by HOCl can be repaired (reduced back to amines) by SCN⁻; (2) cysteine moieties in the active sites of enzymes may not necessarily be over-oxidized (oxidized beyond the disulfide oxidation state) by an excess of HOCl: this is attributed to the hydrophobic environment (oxidation past the disulfide state also involves hydrolysis); (3) SCN⁻ has been shown herein to be able to restore most of the activity of house-keeping enzymes that have been damaged by excess HOCl; (4) post treatment with SCN⁻ can reverse otherwise fatal damage caused by HOCl; (5) because no other reaction of SCN⁻ with oxidized moieties are known (e.g., oxidized sulfur centers, etc.), these results suggest repair of chloramines alone can be sufficient to revive HOCl-damaged cells; and (6) under conditions of severe oxidative stress, SCN⁻ is expected to be over-oxidized (to higher oxides and eventually cyanate and sulfate). However, the Examples demonstrate that reperfusion of SCN⁻ can repair tissue damage that might otherwise result in necrosis.

Thus, in accordance with the present invention, there have been provided compositions, as well as methods of producing and using same that fully satisfy the objectives and advantages set forth hereinabove. Although the invention has been described in conjunction with the specific drawings, experimentation, results and language set forth hereinabove, it is evident that many alternatives, modifications, and variations will be apparent to those skilled in the art. Accordingly, it is intended to embrace all such alternatives, modifications and variations that fall within the spirit and broad scope of the presently disclosed and claimed inventive concept(s).

### REFERENCES

The following references, to the extent that they provide exemplary procedural or other details supplementary to those set forth herein, are specifically incorporated herein by reference.

Ashby et al., J. Am. Chem. Soc., 2004, 126:15976-15977.

Hampton, et al. Blood, 1998, 92:3007.

Nagy et al., Biochemistry, 2006, 45:12610-12616.

Tae et al., Org. Lett., 2009, 11:859-861.

Xulu and Ashby, Biochemistry, 2010, 49:2068-2074.

What is claimed is:

1. A method of producing hypothiocyanite, comprising the steps of:
treating at least one eukaryotic cell with a halogenating agent to produce at least one halogenated eukaryotic cell; and
reacting the at least one halogenated eukaryotic cell with thiocyanate, wherein the thiocyanate reacts with the halogen present on a surface of the at least one eukaryotic cell to produce hypothiocyanite.

2. The method of claim 1, wherein the halogenating agent comprises hypochlorous acid.

3. The method of claim 1, wherein the at least one halogenated eukaryotic cell is further defined as a halogenated eukaryotic tissue.

4. The method of claim 1, wherein the at least one halogenated eukaryotic cell is a foodstuff.

5. The method of claim 4, wherein the foodstuff is selected from the group consisting of animal protein, animal meat, vegetable, fruit, byproducts thereof, and combinations thereof.

6. The method of claim 1, wherein the reaction with thiocyanate substantially removes an odor associated with the halogen present on the surface of the at least one eukaryotic cell.

7. The method of claim 1, wherein the production of hypothiocyanite provides antimicrobial protection to the at least one eukaryotic cell.

8. A method of producing hypothiocyanite, comprising the step of:
reacting at least one halogenated eukaryotic cell with thiocyanate, wherein the thiocyanate reacts with the halogen present on a surface of the at least one eukaryotic cell to produce hypothiocyanite.

9. The method of claim 8, wherein the halogenating agent comprises hypochlorous acid.

10. The method of claim 8, wherein the at least one halogenated eukaryotic cell is further defined as a halogenated eukaryotic tissue.

11. The method of claim 8, wherein the at least one halogenated eukaryotic cell is a foodstuff.

12. The method of claim 11, wherein the foodstuff is selected from the group consisting of animal protein, animal meat, vegetable, fruit, byproducts thereof, and combinations thereof.

13. The method of claim 8, wherein the reaction with thiocyanate substantially removes an odor associated with the halogen present on the surface of the at least one eukaryotic cell.

14. The method of claim 8, wherein the production of hypothiocyanite provides antimicrobial protection to the at least one eukaryotic cell.

15. A method of providing antimicrobial protection to at least one eukaryotic cell, comprising the steps of:
treating at least one eukaryotic cell having immobilized oxidant on a surface thereof with thiocyanate, wherein the at least one eukaryotic cell has been treated with a halogenating agent to provide the immobilized oxidant on the surface thereof, and whereby treatment of the at least one eukaryotic cell having immobilized oxidant on the surface thereof with thiocyanate results in the production of hypothiocyanite.

16. The method of claim 15, wherein the halogenating agent comprises hypochlorous acid, and the immobilized oxidant comprises hypochlorite.

17. The method of claim 15, wherein the at least one eukaryotic cell is further defined as a eukaryotic tissue.

18. The method of claim 15, wherein the at least one eukaryotic cell is a foodstuff.

19. The method of claim 18, wherein the foodstuff is selected from the group consisting of animal protein, animal meat, vegetable, fruit, byproducts thereof, and combinations thereof.

20. The method of claim 15, wherein the reaction with thiocyanate substantially removes an odor associated with the immobilized oxidant present on the surface of the at least one eukaryotic cell.
